(19) United States
(12) Patent Application Publication
(10) Pub. No.: US 2024/0158322 A1
(43) Pub. Date: May 16, 2024

(54) Process and System for Preparing a Target Compound

(71) Applicants: LINDE GMBH, Pullach (DE); Clariant International Ltd., Muttenz (CH)

(72) Inventors: Mathieu Zellhuber, Martinsried (DE); Martin Schubert, München (DE); Andreas Meiswinkel, Rimsting (DE); Wolfgang Muller, München (DE); Ernst Haidegger, Riemerling (DE); Gerhard Mestl, Bruckmühl (DE); Klaus Wanninger, Bruckmühl (DE); Peter Scheck, München (DE)

(21) Appl. No.: 18/549,584

(22) PCT Filed: Mar. 14, 2022
(86) PCT No.: PCT/EP2022/056573

§ 371 Date: Sep. 7, 2023

(30) Foreign Application Priority Data
Mar. 15, 2021 (DE) 102021202500.5

(51) Int. Cl. C07C 5/48; B01J 8/06

(52) U.S. Cl. CPC C07C 5/48; B01J 8/067; B01J 2208/0053; B01J 2208/065; C07C 2523/22; C07C 2523/28

(57) ABSTRACT

A method for producing a target compound includes distributing a feed mixture at a temperature in a first temperature range to a plurality of parallel reaction tubes of a shell-and-tube reactor, and subjecting the feed mixture in first tube sections of the reaction tubes to heating to a temperature in a second temperature range and in second tube sections of the reaction tubes arranged downstream of the first tube sections to oxidative catalytic conversion using one or more catalysts. A gas mixture flowing out of the second tube sections is brought into contact in third tube sections arranged downstream of the second tube sections with a catalyst which has a volumetric activity below the highest volumetric activity of the one or the plurality of catalysts arranged in the second tube sections. A gas mixture from the third tube sections is withdrawn from the shell-and-tube reactor without further catalytic conversion.

### CROSS-REFERENCE TO RELATED APPLICATIONS

This application is the national phase of, and claims priority to, International Application No. PCT/EP2022/056573, filed Mar. 14, 2022, which claims priority to German Patent Application No. DE102021202500.5, filed Mar. 15, 2021.

### FIELD OF THE INVENTION

The invention relates to a method and a plant for producing a target compound.

### BACKGROUND

Oxidative dehydrogenation (ODH) of kerosenes having two to four carbon atoms is generally known. During the ODH, said kerosenes are converted with oxygen, inter alia, to give the respective olefins and water. The invention relates in particular to the oxidative dehydrogenation of ethane to ethylene, hereinafter also referred to as ODHE. However, the invention is in principle not limited to the oxidative dehydrogenation of ethane, but may also extend to the oxidative dehydrogenation (ODH) of other kerosenes such as propane or butane. The following explanations apply accordingly in this case.

ODH(E) may be advantageous over more established methods for producing olefins, such as steam cracking or catalytic dehydrogenation. For instance, due to the exothermic nature of the reactions involved and the practically irreversible formation of water, there is no thermodynamic equilibrium limitation. The ODH(E) can be carried out at comparatively low reaction temperatures. In principle, no regeneration of the catalysts used is required, since the presence of oxygen enables or causes regeneration in situ. Finally, in contrast to steam cracking, lower amounts of valueless by-products, such as coke, are formed.

For further details regarding ODH(E), reference may be made to relevant literature, for example Ivars, F. and López Nieto, J. M., Light Alkanes Oxidation: Targets Reached and Current Challenges, in Duprez, D. and Cavani, F. (eds.), Handbook of Advanced Methods and Processes in Oxidation Catalysis: From Laboratory to Industry, London 2014: Imperial College Press, pages 767-834, or Gartner, C. A. et al., Oxidative Dehydrogenation of Ethane: Common Principles and Mechanistic Aspects, ChemCatChem, vol. 5, no. 11, 2013, pages 3196 to 3217, and X. Li, E. Iglesia, Kinetics and Mechanism of Ethane Oxidation to Acetic Acid on Catalysts Based on Mo-V-Nb Oxides, J. Phys. Chem. C, 2008, 112, 15001-15008.

In particular, MoVNb-based catalyst systems have shown promise for ODH(E), as mentioned, for example, in F. Cavani et al, “Oxidative dehydrogenation of ethane and propane: How far from commercial implementation?”, Catal. Today, 2007, 127, 113-131. Catalyst systems additionally containing Te can also be used. Where reference is made herein to a “MoVNb-based catalyst system” or a “MoVTeNb-based catalyst system”, this shall be understood to mean a catalyst system which has the elements mentioned as a mixed oxide, also expressed as MoVNbOₓ or MoVTeNbOₓ, respectively. Where Te is given in brackets, this indicates its optional presence. The invention is used in particular with such catalyst systems.

During the ODH, particularly when MoVNb(Te)Oₓ-based catalysts are used under industrially relevant reaction conditions, significant amounts of the respective carboxylic acids of the kerosenes used, in particular acetic acid in the case of ODHE, are formed as by-products. For economical plant operation, co-production of olefins and the carboxylic acids is therefore generally unavoidable when using the catalyst type described.

In general, low-oxygen conditions are used in technically relevant oxidative processes such as the production of maleic anhydride (MSA) from butane, butene or benzene or also the two-step synthesis of acrylic acid from propylene via acrolein as an intermediate. Oxygen-depleted air is usually used as oxidant. By way of example, DE 198 37 519 A1 can be mentioned here for the oxidation of propane to acrolein and/or acrylic acid. A current overview of MSA synthesis, which is carried out without exception with air as oxidant, can also be found, for example, in P. V. Mangili et al., “Eco-efficiency and techno-economic analysis for maleic anhydride manufacturing processes”, Clean Technol. Environ. Policy 2019, 21, 1073-1090.

Generally, however, the use of air or oxygen-enriched air as oxidant is also discussed in the context of process intensification. Not least, however, the avoidance of explosive mixtures also plays a role in this context. For this reason, too, air is preferably used as the oxidant.

EP 2 716 621 A1, EP 2 716 622 A1, WO 2018/115416A1, WO 2018/115418A1, WO 2018/082945 A1 and EP 3 339 277 A1 of the applicant disclose the supply of pure oxygen in ODH(E), e.g. oxygen obtained from distillative air separation, as an alternative option in addition to the use of air or oxygen-enriched or oxygen-depleted air, but not the associated special requirements for reaction control and in particular the required coordination of catalyst and reaction control. On the other hand, the provision of oxygen by suitable methods such as distillative air separation or pressure swing adsorption, as already stated in the applications cited above, is an established technology that can be implemented easily and cost-effectively at almost any scale.

However, the use of oxygen as oxidant, in particular in low dilution conditions, then leads to explosive mixtures within the plant. Mixing concepts that can be used here to avoid explosive mixtures in certain plant parts or reactor areas are known and are described, for example, in EP 3 476 471 A1 for a commercial shell-and-tube reactor; however, in addition to structural measures (e.g. the installation of flame or detonation barriers, the minimization of free spaces, pressure-resistant design), these or other approaches presuppose in particular that the corresponding ignition temperature is far from being reached before the actual reactor.

According to the prior art, the ODH(E) is preferably carried out in fixed-bed reactors, in particular in cooled shell-and-tube reactors, e.g. with molten salt cooling. For highly exothermic reaction, i.e. in particular oxidative reactions, which also includes ODH(E), the use of a reactor bed with several zones is generally known. Basic principles are described, for example, in WO 2019/243480 A1 by the applicant. This document discloses the principle that different catalyst beds or corresponding reaction zones, which have different catalyst loadings and/or catalyst activities per spatial unit, are used.

Shell-and-tube reactors typically used for ODH(E) with up to several tens of thousands of parallel tubes in large-scale applications are complex and cost-intensive constructions or apparatuses. Therefore, for both design and cost reasons, the size and dimensions must be as compact as possible. An important parameter here is the length of the individual reaction tube, which must be utilized as efficiently as possible, which means that its length should be kept as short as possible. In particular, the emptying and filling process in the above-mentioned shell-and-tube reactors is also extremely complex, with the result that corresponding shell-and-tube reactors should be operated as conservatively as possible in order to ensure a long service life.

In particular, the end regions of the corresponding (single- or multi-layer) catalyst beds in the individual reaction tubes are exposed to particular stresses, since only a low residual oxygen concentration is usually contained therein as a result of the progress of the reaction. In the following, the term “end” or “end region” or “terminal ends” etc. is to be understood as meaning the region in which the gas flowing through the reactor or a corresponding reaction tube leaves the catalyst bed, i.e. is thereafter no longer subjected to any catalytic conversion in the reactor.

However, the aforementioned catalysts require a certain minimum oxygen content in the reaction gas in order not to be destroyed. On the other hand, the oxygen content at the reactor outlet must not exceed a certain limit value in order to avoid excessive oxygen enrichment and thus the possible formation of an explosive atmosphere in subsequent method steps.

According to the prior art, the use of downstream oxygen removal is known in order to still ensure a sufficient oxygen content at the outlet of the catalyst beds responsible for the conversion to products of value. For example, U.S. Pat. No. 8,519,210 B2 describes catalytic oxygen removal downstream or even integrated into an ODHE reactor, although an “oxygen elimination catalyst” is only mentioned in quite general terms. In the descriptive section, combustion of preferably carbon monoxide and optionally hydrocarbons with two and fewer carbon atoms, which may mean a corresponding yield loss, is set forth therein for oxygen removal. According to this document, it is thus possible in particular to use a material that is independent of and different from the actual ODHE catalyst, and the underlying reaction is a conversion to carbon monoxide and/or carbon dioxide and water.

A similar method is also described in WO 2017/144584 A1. Here, too, the oxygen removal catalyst is preferably used in the ODH reactor downstream of the main reaction zone, wherein the oxygen removal catalyst, although similar to the ODH catalyst, preferably contains additional elements such as Sb, Pt, Pd and Cu or Fe, i.e., preferably has a different composition or is even selected from a different catalyst class. The additional elements mentioned typically also mostly catalyze the conversion to carbon monoxide and/or carbon dioxide and water.

In principle, an enlargement, in particular lengthening, of the catalyst bed for the purpose of targeted “overdimensioning” is also conceivable in order to ensure a long service life even if the catalyst is destroyed or deactivated due to oxygen deficits at the reactor outlet. In this case, in principle, a somewhat lower temperature can be selected at the start of run (SOR) in order to compensate for the deactivation by increasing the temperature. However, in particular due to the effect on the length of the catalyst bed, but also possibly due to a loss of selectivity associated with the deactivation, such a measure is only of limited practical use and should therefore be avoided.

Avoiding an “overdimensioned” catalyst filling additionally leads to a reduction in pressure drop across the reactor due to the associated reduction in length of the individual reaction tubes, which is significant in particular for “low-pressure” methods such as ODH(E) (typically performed at less than 10 bar (abs.) or less than 6 bar (abs.)).

### SUMMARY

According to the invention, therefore, an object is to keep the time interval between two catalyst changes as long as possible while at the same time achieving economical production during the run time, i.e., to achieve the highest possible and most constant selectivity or yield of products of value. To this end, it is necessary to minimize adjustments to the reaction conditions, such as, for example the reaction temperature. It is therefore necessary to achieve stabilization of the reaction conditions.

According to one embodiment of the invention, a method for producing a target compound includes distributing a feed mixture at a temperature in a first temperature range to a plurality of parallel reaction tubes of a shell-and-tube reactor. The feed mixture is subjected in first tube sections of the reaction tubes to heating to a temperature in a second temperature range, and is subjected in second tube sections of the reaction tubes arranged downstream of the first tube sections to oxidative catalytic conversion using one or more catalysts arranged in the second tube sections. A gas mixture flowing out of the second tube sections is brought into contact in third tube sections arranged downstream of the second tube sections with a catalyst arranged in the third tube sections which has a volumetric activity below the highest volumetric activity of the one or the plurality of catalysts arranged in the second tube sections. A gas mixture flowing out of the third tube sections is withdrawn from the shell-and-tube reactor without further catalytic conversion.

According to another embodiment of the invention, a plant for producing a target compound includes a shell-and-tube reactor which has a plurality of parallel reaction tubes having first tube sections and second tube sections arranged downstream of the first tube sections. One or more catalysts are arranged in the second tube sections. The plant further has means configured to: distribute a feed mixture at a temperature in a first temperature range to the reaction tubes; subject said feed mixture to heating to a temperature in a second temperature range; and subject said feed mixture to an oxidative catalytic conversion in the second tube sections using the one or the more plurality of catalysts arranged in the second tube sections. The second tube sections are fluidically connected to third tube sections arranged downstream of the second tube sections. In the third tube sections, a catalyst is arranged which has a volumetric activity below the highest volumetric activity of the one or the plurality of catalysts arranged in the second tube sections. Downstream of the third tube sections, no further catalysts are provided in the shell-and-tube reactor.

### BRIEF DESCRIPTION OF THE DRAWINGS

FIG. 1 illustrates different catalyst activities for catalysts obtained at different calcination temperatures.

FIG. 2 illustrates a plant according to an embodiment of the invention in a simplified schematic illustration.

FIG. 3 illustrates a reactor according to an embodiment of the invention in a simplified schematic illustration.

### WRITTEN DESCRIPTION

In one embodiment, the invention now makes use of the fact that the activity, also expressed in particular as volumetric activity hereinafter, of a particular catalyst material can be influenced by the production and in particular by a single production step. It was found, in particular for the advantageously used MoVNb(Te)Oₓ catalysts, that the calcination conditions have a direct influence on their respective activity. The catalytically active material itself remains in principle the same in terms of composition and can in particular be obtained from the same synthesis approach.

The lower volumetric activity is usually accompanied by a lower pore volume and/or a lower BET surface area. The BET surface area is the mass-specific surface area, which is calculated from experimental data according to known methods and usually expressed in the unit square meter per gram (m²·g¹). The BET measurement is known to the person skilled in the art from relevant textbooks and standards, for example DIN ISO 9277:2003-05, “Determination of the specific surface area of solids by gas adsorption using the BET method (ISO 9277:1995)”. However, this is not a necessary requirement for the implementation of the invention, but relates to a possible embodiment. The specific pore volume of a catalyst can be determined, for example, by means of nitrogen physisorption measurements.

In the aforementioned embodiment, the invention makes use of this by employing a catalyst of advantageously the same type and elemental composition with lower activity (i.e., a “more inert” catalyst) in terminal zones of the reaction tubes of a shell-and-tube reactor, which can be formed in this way as a “polishing” zone. As explained below, fluctuations in the oxygen content at the outlet from the main reaction zones can be compensated for in this way.

In principle, however, a catalyst produced in a different way and having a reduced activity can also be used in the context of the invention. Specific examples are mentioned below.

Overall, the invention proposes a method for producing a target compound, in which a feed mixture at a temperature in a first temperature range is distributed to a plurality of parallel reaction tubes of a shell-and-tube reactor, is subjected in first tube sections of the reaction tubes to heating to a temperature in a second temperature range, and is subjected in second tube sections of the reaction tubes arranged downstream of the first tube sections to oxidative catalytic conversion using one or more catalysts arranged in the second tube sections. According to the invention, a gas mixture flowing out of the second tube sections is brought into contact in third tube sections arranged downstream of the second tube sections, the aforementioned “polishing zone” or a corresponding “polishing bed”, with a catalyst arranged in the third tube sections which has a volumetric activity below the highest volumetric activity of the one or the plurality of catalysts arranged in the second tube sections. A gas mixture flowing out of the third tube sections is withdrawn from the shell-and-tube reactor without further catalytic conversion. The catalyst beds provided for catalytic conversion in the third tube sections are thus the terminal catalyst beds of the shell-and-tube reactor.

The disadvantages mentioned at the outset can be overcome by using the invention. A substantial advantageous effect of the polishing zone used in the third tube sections according to the invention is that any fluctuations in the oxygen content at the outlet from the main reaction zones, i.e. the second tube sections, can be compensated for. Therefore, although the catalyst in this downstream polishing zone may use the same catalytically active material as in the preceding main reaction zone(s), it is preferably always such that it is relatively inert and thus insensitive to possible (sudden) changes in oxygen concentration. Nevertheless, ethane continues to be converted in this zone by oxidative dehydrogenation to the main product ethylene and the by-product acetic acid. Carbon oxides continue to be formed only in minor amounts. This means that selective value creation continues to take place and non-selective oxidation to carbon oxides and water, as is the case with catalysts for oxygen elimination known from the prior art, is avoided as much as possible.

For further advantages, reference is made to the explanation of the objects of the invention, which are at least partially achieved by the proposed measures.

The catalyst arranged in the third tube sections and the one or at least one of the plurality of catalysts arranged in the second tube sections advantageously contain at least the metals molybdenum, vanadium, niobium and optionally tellurium, in particular in the form of a corresponding mixed oxide, since, as has been demonstrated in accordance with the invention, the aforementioned advantageous effects are particularly pronounced with corresponding catalysts.

The catalyst arranged in the third tube sections and the one or at least one of the plurality of catalysts arranged in the second tube sections can furthermore be produced according to the invention at least partially from the oxides of the corresponding metals. The catalyst production is therefore extremely cost-effective due to the readily available starting materials.

The catalyst arranged in the third tube sections and the one or at least one of the plurality of catalysts arranged in the second tube sections advantageously have an identical elemental composition, as already discussed. This enables simple production of the corresponding catalysts, the differences between which are merely due to the different manufacturing process. According to the understanding applied here, an “identical elemental composition” should still be present even if the contents of the individual elements or their compounds do not differ by more than 10%, 5% or 1% between the different catalysts.

Advantageously, in one embodiment of the invention, the catalyst arranged in the third tube sections has an activity which is at least 10% lower than the one or at least one of the plurality of catalysts arranged in the second tube sections due to different calcination intensities. The activity can also be at least 20%, 30% or 40% lower, for example. A calcination intensity is in particular conditioned by the calcination procedure, i.e. the technology used in the calcination, but also certain parameters thereof, for example particularly intensive calcination, e.g. calcination that is long-lasting or carried out at an elevated temperature.

In a further embodiment of the invention, a less active catalyst with essentially the same composition for use in the third tube sections can also be a spent and thus aged catalyst, i.e., for example, a catalyst from a more active zone, in particular from the zone with the highest activity, which has reached the minimum service life in a corresponding reactor. Usually, during the run time, typically considerably more than 1 year and up to several years in the case of oxidative methods such as ODH(E), there is a gradual deactivation of the catalyst or reduction in the catalyst activity, which is usually compensated for by increasing the temperature. In a catalyst exchange, however, the catalyst, in particular the portion with the highest volumetric activity in a staged bed, is not deactivated to such an extent that it no longer has any activity at all. Rather, the activity is only reduced to such an extent that a further increase in temperature can only be achieved through considerable effort in the plant periphery. Therefore, within the scope of the invention, a spent catalyst can thus also be used for the polishing zone. In this way, a portion of the spent catalyst can be directly recycled, which saves costs in the disposal or recycling of spent catalyst or costs in the procurement/production of specially manufactured catalyst for the polishing bed.

To manage fluctuating oxygen contents at the end of the second tube sections, a catalyst with the same catalytically active material can therefore be used in whole or in part in the third tube sections in accordance with the invention. In this case, however, a material is advantageously selected that has a lower activity and is therefore extremely inert.

According to the invention, the downstream polishing zone formed by the third tube sections, in which, indeed, a less active catalyst is used, has no appreciable influence on the overall reactor performance, i.e. the performance of the totality of the reactive beds (one or more main reaction zones and polishing zone), in terms of conversion and selectivity to commercial products, since, indeed, the predominant conversion occurs in the (single- or multi-layer) catalyst bed of the main reaction zone (i.e. the second tube sections). Consequently, adjustment of the total activity by increasing the temperature remains possible, but may be carried out with a significant time delay or with a reduced gradient by means of the invention. Thus, stabilization of the reaction conditions over time is again achieved.

The length of the polishing zone is preferably at least ten times the equivalent diameter of a catalyst particle used, but preferably less than 40 cm or less than 30 cm, particularly preferably between 5 and 25 cm. In addition, a design in which the length of the downstream polishing zone in relation to the one or more main reaction zones is less than 0.1 in total, preferably less than 0.07, and particularly preferably less than 0.04, is in particular relevant for the technical design.

In other words, a length of a region in which the catalyst is arranged in the third tube sections is less than 40 cm in absolute dimensions and/or this length is less than 0.1 relative to a total length of a region in which the one or the plurality of catalysts is or are arranged in the second tube sections.

In particular, in summary again, the embodiment of the catalysts according to the invention may be such, due to being manufactured differently, that a volumetric activity in the third tube sections is below a maximum volumetric activity in the second tube sections.

In a further embodiment of the invention, a catalyst may also be used in the polishing zone (i.e., the third tube sections) which, although similar to that of the main reaction zones (i.e., the second tube sections) according to the preceding statements, is specifically optimized for gas composition near the outlet (i.e., in particular, a comparatively high ethylene and low oxygen content). Adjustable variables (e.g. composition, variables obtained by means of BET analysis and the pore volume) are set out below.

Physically measurable distinguishing features for the catalysts used can optionally be derived, for example, in particular (but not conclusively) from the BET analysis known to the person skilled in the art and/or the pore volume.

Thus, in particular, a pore volume and/or a BET surface area in the third tube sections may be below, in particular 15 to 60% below, a maximum pore volume and/or below a maximum BET surface area in the second tube sections. As an alternative to the aforementioned use of MoVNbTeOₓ as in the main reaction zones (i.e., the second tube sections), it is also possible in particular to use a catalyst that differs in part from the material in the main reaction zones. For example, it can be a catalyst of the MoVNbOₓ type (i.e. without Te).

However, a different active material in particular can also be used in the downstream polishing zone. Thus, the overall bed layout can be further optimized by a combination of dilution/reactor cooling and modified active material.

As mentioned, the invention can be used in particular in connection with an ODH of alkanes such that the feed mixture advantageously contains oxygen and a kerosene, in particular having two to six carbon atoms, and the oxidative conversion is performed as an oxidative dehydrogenation of the kerosene. In an ODHE employed with particular advantages, ethane is used as the kerosene and an oxidative dehydrogenation of ethane is performed.

The oxidative conversion is advantageously carried out at a temperature of the catalyst in a range between 240 and 500° C., preferably between 280 and 450° C., in particular between 300 and 400° C.

The feed mixture is advantageously fed to the reactor at a pressure in a pressure range from 1 to 10 bar (abs.), in particular from 2 to 6 bar (abs.). This is therefore a method operating at comparatively low pressure.

Particularly advantageously, within the scope of the invention, a water content can be set in the feed mixture which can be between 5 and 95 vol %, particularly between 10 and 50 vol %, and further particularly between 14 and 35 vol %. As also disclosed, for example, in EP 3 558 910 B1 to the applicant, it is also possible, for example, to determine at least one characteristic variable indicating an activity of the or one of the catalysts and, on this basis, to set an amount of water in the reaction feed flow on the basis of the at least one determined characteristic variable.

In particular, an embodiment in which the feed mixture contains ethane and in which the molar ratio of water to ethane in the feed mixture is at least 0.23 may be advantageous.

The invention can be applied regardless of how the cooling medium is guided (i.e., in co-current or counter-current). Likewise, different cooling circuits in combination with different catalyst layers are conceivable (as also indicated in more detail still in WO 2019/243480 A1).

There is a particular advantage if the reactor is designed in such a way that the reactor in the region of the polishing zone, i.e. the third tube sections, is explicitly additionally cooled in a different way, i.e. in said region there is the option of a separate cooling circuit (possibly even with a different coolant flow direction). The advantage of this is targeted temperature adjustment and thus activity adjustment in the reactive polishing zone. As a result, this zone can, for example, also be explicitly “switched on” by corresponding heat input, or “switched off” if not required or only required to a small extent.

In other words, the invention proposes in one embodiment that the reaction tubes are cooled using one or more cooling media flowing around the reaction tubes. The first tube sections, the second tube sections and the third tube sections can in this case be cooled particularly advantageously using different cooling media, the same cooling medium in different cooling media circuits, and/or the same or different cooling media in different or the same flow directions.

The invention also relates to a plant for producing a target compound, having a shell-and-tube reactor which has a plurality of parallel reaction tubes having first tube sections and second tube sections arranged downstream of the first tube sections, wherein one or more catalysts are arranged in the second tube sections, and the plant has means configured to distribute a feed mixture at a temperature in a first temperature range to the reaction tubes, to subject said feed mixture to heating to a temperature in a second temperature range, and to subject said feed mixture to an oxidative catalytic conversion in the second tube sections using the one or the plurality of catalysts arranged in the second tube sections.

According to the invention, the second tube sections are fluidically connected to third tube sections arranged downstream of the second tube sections, wherein in the third tube sections a catalyst is arranged which has a volumetric activity below the highest volumetric activity of the one or the plurality of catalysts arranged in the second tube sections, and wherein downstream of the third tube sections no further catalysts are provided in the shell-and-tube reactor.

For further features and advantages of the plant proposed according to the invention, reference is expressly made to the above explanations. In corresponding embodiments, the plant is in this respect configured in particular to perform a method as has already been explained above, likewise in various embodiments. The explanations apply accordingly.

In summary, the use of the downstream polishing zone according to the invention, i.e., the embodiment with the third tube sections, provides advantages comprising an increase in the run time of the main catalyst bed, an increase in the tolerance of the main catalyst bed to disruptions such as deviations in temperature, flow and composition (in particular the oxygen content), a reduction or minimization of temperature adjustments over the run time (i.e., stabilization of reaction conditions) and thus minimization of a decrease in selectivity/yield over time, and improved assurance and stabilization of a maximum acceptable oxygen concentration at the reactor outlet.

The invention is further explained below with reference to examples corresponding to embodiments of the invention and comparative examples not in accordance with the invention, as well as associated figures and tables.

In embodiments, as mentioned, the invention utilizes the fact that the activity of a particular catalyst material can be influenced by the production. The catalytically active material itself remains in principle the same in terms of composition and can in particular be obtained from the same synthesis approach. This surprising effect was found in a catalytic test of MoVNb(Te)Oₓ catalyst material with the same synthesis approach and thus the same stoichiometry (element composition), but different calcination temperatures.

In this context, the catalyst material can in principle be produced as described in DE 10 2017 000 861 A1 in Example 2. Here, the suitable metal oxides in each case can be subjected to hydrothermal synthesis.

In the method used in DE 10 2017 000 861 A1, which can also be used within the scope of the invention, TeO₂ was slurried in 200 g of distilled water and ground in a planetary ball mill with 1 cm balls (ZrO₂). The portion was then transferred to a beaker with 500 mL of distilled water. Nb₂O₅ was slurried in 200 g of distilled water and ground in the same ball mill. The portion was then transferred to a beaker with 500 mL of distilled water. The next morning, the temperature was raised to 80° C., and 107.8 g of oxalic acid dihydrate was added to the Nb₂O₅ suspension, which was stirred for about 1 h. 6 L of distilled water was placed in an autoclave (40 L) and heated to 80° C. with stirring (stirrer speed 90 rpm). When the water reached the temperature, 61.58 g of citric acid, 19.9 g of ethylene glycol, 615.5 g of MoO₃, 124.5 g of V₂O₅, the ground TeO₂ and the ground Nb₂O₅ in oxalic acid were added successively. 850 mL of distilled water was used to transfer and rinse the vessels. The total amount of water in the autoclave was 8.25 L. Nitrogen was then added on top. Hydrothermal synthesis was performed in a 40 L autoclave at 190° C./48 h. After synthesis, filtering was performed using a vacuum pump with blue sand filter and the filter cake was washed with 5 L of distilled water.

Drying was carried out at 80° C. in a drying oven for 3 days and then the product was ground in an impact mill. A solid yield of 0.8 kg was obtained. Subsequent calcination was carried out at 280° C. for 4 h in air (heating rate 5° C./min air: 1 L/min). Activation was carried out in a retort at 600° C. for 2 h (heating rate 5° C./min nitrogen: 0.5 L/min).

However, unlike the method described above, the graduated calcination temperatures listed in Table 1 were used. Furthermore, the catalysts listed in Table 1 were activated in a rotary kiln rather than in the retort. The catalysts obtained are denoted as 1 to 3. The specific surface area according to BET as given in Table 1 and the pore volume refer to the calcined catalyst material before tabletting.

TABLE 1

| Catalyst sample | Cat. 1 | Cat. 2 | Cat. 3 |
|---|---|---|---|
| Calcination temperature of the catalyst [° C.] | 630 | 650 | 670 |
| Specific surface area (according to BET) [m²/g] | 11 | 9.8 | 7.1 |
| Specific pore volume [cm³/g] | 0.0533 | 0.0405 | 0.0293 |
| Reaction temperature window [° C.] | 230-295 | 270-300.5 | 295-310 |
| Ethane conversion range, measured for the reaction | 4.4-47.5 | 17.9-46.2 | 30.0-43.9 |
| temperature window [%] | | | |

The catalysts produced in this way were tested with respect to their activity in a test plant 1 under exactly identical conditions (filled catalyst amount of 46 g, system pressure of 3.5 bar (abs.), composition of the reaction feed of ethane to oxygen to water (vapor) of 55.3 to 20.7 to 24 (in each case mol %), GHSV of 1140 (NL_{gas}/h)/L_{catalyst}). The corresponding experimental reactor (usable length 0.9 m, inner diameter of reaction chamber 10 mm) is designed as a double tube. The heating or cooling is carried out with the aid of a thermal oil bath, wherein the thermal oil is pumped through the outer chamber of the reactor and thus heats or also simultaneously cools the inner chamber/reaction zone (the conversion is an exothermic reaction). At an oil bath temperature of 295° C., clear absolute and relative activity gradations of +21% and −23% (relative in each case) were found for the differently calcined catalysts compared with the base case (standard calcination temperature of 650° C.).

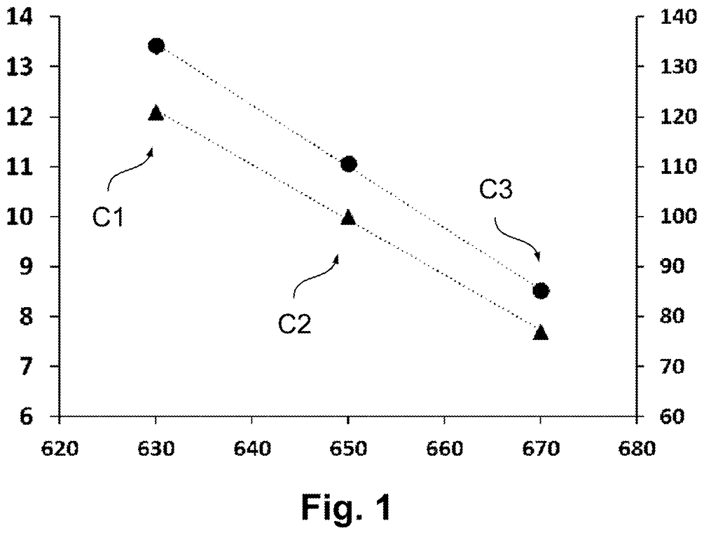

The activity gradations are illustrated in FIG. 1, in which the activity in the form of ethane conversion in moles per liter of catalyst and hour (i.e., the activity per catalyst volume) is illustrated on the left vertical axis (circles in the diagram) and the relative activity in percent is illustrated on the right vertical axis (triangles in the diagram) in relation to the calcination temperature on the horizontal axis.

The values obtained for the respective catalysts or catalyst samples according to Table 1 are shown as C1, C2 and C3.

On the basis of the observed trend in the activities of catalysts 1 to 3 as a function of the calcination temperature (cf. FIG. 1 and Table 1), it can be assumed that the activity of the catalysts can be further influenced (increased or reduced) as a function of the calcination temperature, at least within certain limits, as long as (also given the same calcination technology) the calcination temperature and duration, i.e. the calcination intensity, is set in such a way that either a solid/crystal phase sufficiently stable for catalysis is formed or the solid/crystal phase is not damaged by excessively high calcination intensity.

The findings according to the invention explained above are surprising. The different activity behavior of the catalyst samples according to the invention can surprisingly be correlated with the data from the catalyst characterization (cf. Table 1 and 2). By selecting the calcination temperature during catalyst production, an influence on the specific surface area, and, even more significantly, the specific pore volume (and thus the activity) can be achieved as a novel finding.

While a different activity usually also has a fairly strong influence on selectivity (generally, a higher activity is accompanied by reduced selectivity), surprisingly, within the scope of the invention, high or even constantly high selectivity of the overall reaction bed can be achieved.

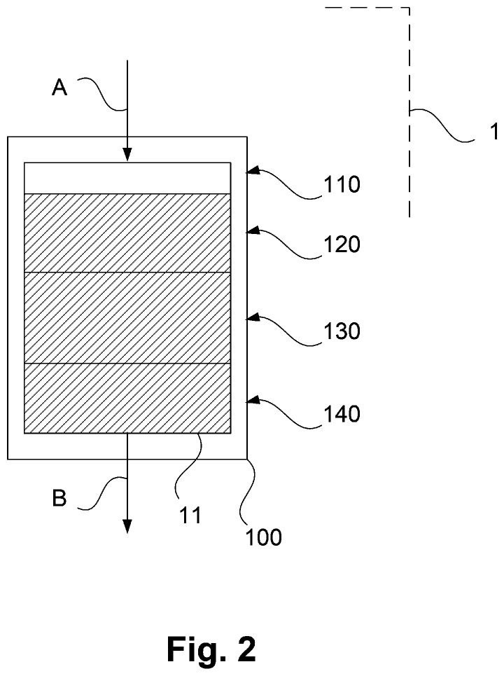

FIG. 2 illustrates a plant for producing olefins in accordance with an embodiment of the invention in the form of a highly simplified plant diagram that is designated overall by 1. The plant 1 is only indicated schematically in this case. In particular, the basic arrangement of the reaction zone and the polishing zone is illustrated using a greatly enlarged reaction tube 11, not drawn to scale, in a shell-and-tube reactor 100. Although a plant 1 for ODHE is described below, as mentioned, the invention is also suitable for use in ODH of higher hydrocarbons. In this case, the following explanations apply accordingly.

As mentioned, the plant 1 has a shell-and-tube reactor 100 to which, in the example shown, a feed mixture A containing ethane and obtained in any manner is fed. The feed mixture A may contain, for example, hydrocarbons withdrawn from a rectification unit not shown. The feed mixture A can also be preheated, for example, and treated in another way. The feed mixture A may already contain oxygen and, optionally, a reaction moderator such as water vapor, but corresponding media may also be added upstream or in the shell-and-tube reactor 100, as is not separately illustrated. A product mixture B is withdrawn from the tubular reactor 100.

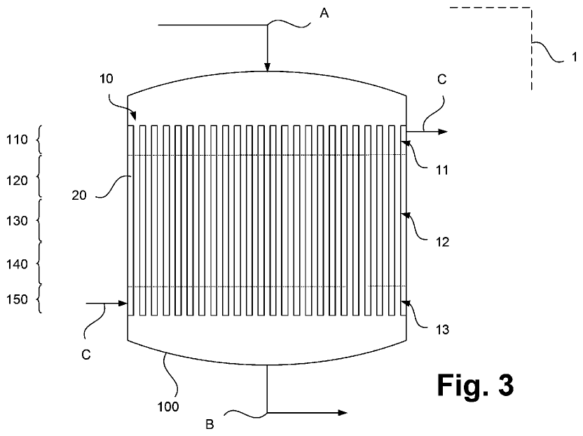

The shell-and-tube reactor 100, shown in detail in FIG. 3, has a plurality of parallel reaction tubes 10 (only partially designated) which extend through a preheating zone 110, then through a plurality of reaction zones 120, 130, 140, three in the example shown, and finally through a polishing zone 150 of the type explained above. The reaction tubes 10 are surrounded by a jacket region 20 through which, in the example, a coolant C of the type explained is guided. The illustration is greatly simplified because, as mentioned, the reaction tubes 10 may be cooled using a plurality of cooling media flowing around the reaction tubes 10, or different tube sections may be cooled using different cooling media, the same cooling media in different cooling media circuits, and/or the same or different cooling media in the same or different flow directions.

After being fed into the shell-and-tube reactor, the feed mixture A is suitably distributed to the reaction tubes 10 at a temperature in a first temperature range. The reaction tubes each have first tube sections 11 located in the preheating zone 110 and second tube sections 12 located in the reaction zones 120, 130 and 140. Third tube sections 13 are located in the polishing zone 150.

Heating is carried out in the first tube sections 11 of the reaction tubes 10, and in the second tube sections 12 of the reaction tubes 10 arranged downstream of the first tube sections 11, the correspondingly preheated feed mixture A is subjected to oxidative catalytic conversion using one or more catalysts arranged in the second tube sections 12.

A gas mixture flowing out of the second tube sections 12 is brought into contact in the third tube sections 13 arranged downstream of the second tube sections 12 with a catalyst arranged in the third tube sections 13 which has a volumetric activity below the highest volumetric activity of the one or the plurality of catalysts arranged in the second tube sections 12, and a gas mixture flowing out of the third tube sections 13 is withdrawn from the shell-and-tube reactor 100 without further catalytic conversion.

Subsequent method steps or plant components are not illustrated. In particular, the process gas can be brought into contact with wash water or a suitable aqueous solution, as a result of which the process gas can be cooled and acetic acid can be washed out of the process gas. The process gas, which is at least largely freed of water and acetic acid, may be further treated and undergo separation of ethylene. Ethane contained in the process gas can be recycled into the reactor 100.

1. A method for producing a target compound, comprising:
distributing a feed mixture at a temperature in a first temperature range to a plurality of parallel reaction tubes of a shell-and-tube reactor;
subjecting the feed mixture in first tube sections of the reaction tubes to heating to a temperature in a second temperature range; and
subject the feed mixture in second tube sections of the reaction tubes arranged downstream of the first tube sections to oxidative catalytic conversion using one or more catalysts arranged in the second tube sections;
wherein:
a gas mixture flowing out of the second tube sections is brought into contact in third tube sections arranged downstream of the second tube sections with a catalyst arranged in the third tube sections which has a volumetric activity below the highest volumetric activity of the one or the plurality of catalysts arranged in the second tube sections; and
a gas mixture flowing out of the third tube sections is withdrawn from the shell-and-tube reactor without further catalytic conversion.

2. The method according to claim 1, in which a volumetric activity in the third tube sections is below a maximum volumetric activity in the second tube sections.

3. The method according to claim 2, in which a pore volume and/or a BET surface area in the third tube sections is below a maximum pore volume and/or below a maximum BET surface area in the second tube sections.

4. The method according to claim 1, in which the catalyst arranged in the third tube sections has an activity which is at least 10% lower than the one or at least one of the plurality of catalysts arranged in the second tube sections due to different calcination intensities.

5. The method according to claim 1, in which a length of a region in which the first catalyst is arranged in the first tube sections is less than 40 cm and/or relative to a total length of a region in which the one or the plurality of catalysts are arranged in the second tube sections is less than 0.1.

6. The method according to claim 1, in which the catalyst arranged in the third tube sections and the one or at least one of the plurality of catalysts arranged in the second tube sections contain at least the metals molybdenum, vanadium, and niobium.

7. The method according to claim 6, in which the catalyst arranged in the third tube sections and the one or at least one of the plurality of catalysts arranged in the second tube sections are at least partially produced from the oxides of the metals.

8. The method according to claim 1, in which the catalyst arranged in the third tube sections and the one or at least one of the plurality of catalysts arranged in the second tube sections have an identical elemental composition.

9. The method according to claim 1, in which the catalyst arranged in the third tube sections is a spent catalyst that was previously used in the second tube sections of the same or a further shell-and-tube reactor.

10. The method according to claim 1, in which the feed mixture contains oxygen and a kerosene, and in which the oxidative conversion is performed as oxidative dehydrogenation of the kerosene.

11. The method according to claim 1, in which the first temperature range is 200 to 280° C. and/or in which the second temperature range is 280 to 450° C..

12. The method according to claim 1, in which the feed mixture contains a water content that is set between 5 and 95 vol %, and wherein the molar ratio of water to ethane in the feed mixture is in particular at least 0.23.

13. The method according to claim 1, in which the reaction tubes are cooled using one or more cooling media flowing around the reaction tube.

14. The method according to claim 13, in which the first tube sections, the second tube sections, and/or the third tube sections are cooled using different cooling media, the same cooling medium in different cooling media circuits, and/or the same or different cooling media in different or the same flow directions.

15. A plant for producing a target compound, having:
a shell-and-tube reactor which has a plurality of parallel reaction tubes having first tube sections and second tube sections arranged downstream of the first tube sections, wherein one or more catalysts are arranged in the second tube sections; and
means configured to:
distribute a feed mixture at a temperature in a first temperature range to the reaction tubes;
subject said feed mixture to heating to a temperature in a second temperature range; and
subject said feed mixture to an oxidative catalytic conversion in the second tube sections using the one or the more plurality of catalysts arranged in the second tube sections;
wherein:
the second tube sections are fluidically connected to third tube sections arranged downstream of the second tube sections;
in the third tube sections a catalyst is arranged which has a volumetric activity below the highest volumetric activity of the one or the plurality of catalysts arranged in the second tube sections;
downstream of the third tube sections no further catalysts are provided in the shell-and-tube reactor.

16. The method according to claim 6, in which the catalyst arranged in the third tube sections and the one or at least one of the plurality of catalysts arranged in the second tube sections further contains tellurium.

17. The method according to claim 16, in which the catalyst arranged in the third tube sections and the one or at least one of the plurality of catalysts arranged in the second tube sections are at least partially produced from the oxides of the metals.

18. The method according to claim 10, in which the kerosene is ethane, and in which the oxidative conversion is performed as oxidative dehydrogenation of ethane.

19. The method according to claim 11, in which the first temperature range is 240 to 260° C., and/or in which the second temperature range is 300 to 400° C.
